(19) United States
(12) Patent Application Publication
(10) Pub. No.: US 2019/0111139 A1
(43) Pub. Date: Apr. 18, 2019

(54) VITAMIN PREPARATION

(71) Applicant: Dantor Biotech AG, Hünenberg (CH)

(72) Inventor: Sven Reichwagen, Ahlen (DE)

(73) Assignee: Dantor Biotech AG, Hunenberg (CH)

(21) Appl. No.: 16/094,659

(22) PCT Filed: Apr. 21, 2017
(86) PCT No.: PCT/EP2017/059496

§ 371 Date: Oct. 18, 2018

(30) Foreign Application Priority Data
Apr. 22, 2016 (DE) 10 2016 107 563.9

(51) Int. Cl. A61K 47/34; A61K 9/00; A61K 31/714; A61K 47/18; A61K 47/26; A61K 9/06; A61K 47/10; A61K 47/38; A61K 47/12; A61K 47/02; A61K 9/70

(52) U.S. Cl. CPC A61K 47/34; A61K 9/0014; A61K 31/714; A61K 47/18; A61K 47/26; A61K 9/7007; A61K 47/10; A61K 47/38; A61K 47/12; A61K 47/02; A61K 9/0043; A61K 9/06

(57) ABSTRACT

The summary relates to a vitamin preparation, especially in gel form, for application to the skin, said preparation containing
a physiologically and/or therapeutically effective amount of vitamin B12,
a poloxamer as well as
customary carrier substances for the formation of a carrier matrix.

The invention relates to a vitamin preparation, particularly in gel form for application to the skin, said preparation containing a physiologically and/or therapeutically effective amount of vitamin B12.

Vitamin B12 belongs to the group of cobalamins and is an essential vitamin the human body is unable to synthesize. The human need in this respect must therefore be satisfied through food. Cobalamins in sufficient amount are especially found in animal-derived foods. However, their origin is always of bacterial nature.

As a rule, vitamin B12 is understood to mean cyanocobalamin. Additional forms are, for example, aquocobalamin, hydroxycobalamin, and methylcobalamin that is usually found in somatic cells. Functioning and being effective as coenzyme B12 is 5′-deoxyadenosylcobalamin. The active forms methylcobalamin and adenosylcobalamin are formed in the body from the other forms.

Within the meaning of the invention especially cyanocobalamin which is customarily referred to as vitamin B12 shall be understood to be “vitamin B12” but also the other precursor forms of the coenzyme B12 as they are indicated above.

For the treatment of a number of skin diseases vitamin B12 has proven to be very effective. It is applied in ointment form for a number of dermatoses, and also for treating atopic eczema. A synthetic or vegetable oil or fat, in particular avocado oil, serves as ointment basis.

Although with customary ointments on oil or fat basis the respective active agent can be satisfactorily spread on the skin portions to be treated, their distribution, however, is only superficial. An appreciable transdermal effect cannot be produced.

From WO 2014/016277 A2 a vitamin preparation is known, by means of which vitamin B12 can be administered transdermally with the help of polysorbates as an entrainer.

It is, therefore, the objective of the present invention to propose a vitamin preparation which is capable of efficiently introducing vitamin B12 also into the upper skin layers where a sustainable effect can then be brought about.

This objective is achieved with a vitamin preparation of the kind first mentioned above that contains a physiologically and/or therapeutically effective amount of vitamin B12, a poloxamer as well as the usual carrier substances needed to establish a carrier matrix.

Poloxamers are block copolymers of ethylene oxide and propylene oxide in which a propylene oxide block is flanked on both sides by ethylene oxide blocks. Poloxamers are surface-active since the polypropylene oxide part is rather hydrophobic, whereas the two polyethylene oxide parts are hydrophilic. Accordingly, they can be classified as non-ionic surfactants that are frequently employed for dispersion and emulsification uses in the chemical-technical industry. Due to their amphiphilic properties they are widely used as carrier materials in cosmetic and pharmaceutical products. Depending on the degree of ethoxilation, poloxamers are present in liquid or solid form.

Poloxamers of different molecular weights and alkylene oxide distributions are known. The proportion of ethylene oxide units is responsible for the hydrophilicity. Common types used in the pharmaceutical and cosmetic industries are poloxamer 188, 237, 338 and 407. The final digit of the numeric coding multiplied by 10 represents the percentage ethylene oxide content. Poloxamer 407 has an ethylene oxide content of 70% by weight.

It has now been surprisingly found that poloxamers, due to their amphiphilic properties, are not only suitable for use as carriers for a wide variety of substances but are also capable of transdermally administering active agents. They act as entrainers which transport the respective active substance, in particular vitamin B12 or its relevant precursors, through the skin or mucous membrane.

Compared to polysorbate 20 mentioned in publication WO 2014/016277 referred to hereinbefore, there is an improved entrainment effect of approx. 20%.

Aside from vitamin B12, other active substances can also be transported by poloxamers through the skin or mucous membrane. This applies in particular to secondary plant substances such as nicotine, caffeine, simple and polyphenols (for example epigallocatechin gallate) and flavonoids. Simple phenols and polyphenols are well known for their anti-inflammatory effect.

The entrainment effect in transdermal preparations containing vitamin B12 can on the whole be further improved if also a polysorbate, in particular polysorbate 20 (Tween®), is present in addition to a poloxamer.

Poloxamers that are preferred according to the present invention are the poloxamers 188 and 407, poloxamers with 80 and 70% by weight of glycol respectively.

As polysorbates the customary and commercially available products may be employed. These are multiply ethoxylated sorbitan fatty acid esters of various fatty acids, for example of lauric acid, palmitic acid, stearic acid, oleic acid or isostearic acid. Preferred polysorbate is polysorbate 20 which is commercially available under the name of Tween®. The amount of polysorbate in a preparation conforming to the invention may be between 20 and 100 mg per 100 g of the preparation and shall in particular be approximately 40 mg per 100 g of the preparation.

In addition to vitamin B12 or another precursor of the coenzyme B12, the vitamin preparation proposed by the present invention contains customary carrier substances to enable a carrier matrix, in particular a gel matrix, to be formed. Such substances are, for example, glycerin, hydroxyethyl cellulose, urea, and demineralized water. For the pH adjustment, preferably to pH 5.4, citric acid and/or caustic soda solution may be employed, which have been shown to be highly biocompatible. However, other acids and bases may be used as well. The preparation may also be provided in the form of a lotion, cream, ointment, spray, tincture or as shampoo.

The inventive vitamin preparation contains vitamin B12 or another form of cobalamin in an amount ranging between 40 and 100 mg, in particular in an amount of 70 mg per 100 g of the preparation. Poloxamer is present in the preparation in an amount ranging between 20 and 500 mg per 100 g of the preparation, in particular in an amount of 100 mg.

In particular, hydroxyethyl cellulose 400 in an amount of between 1000 mg and 5000 mg per 100 g of the preparation is used for gel formation. Further constituents may be glycerin in amount of between 1000 mg and 5000 mg per 100 g as well as urea in an amount ranging between 2000 mg and 7000 mg per 100 g.

Aside from the above mentioned ingredients the preparation may also contain an antiseptic agent, for example polyhexanide. This shall be added in the form of a commercially available solution concentrate (20% m/v) and is preferably present in an amount ranging between 100 and 500 mg of concentrate per 100 g. The addition of polyhexanide may be especially helpful in the event of inflamed and inflammable skin diseases during which attacks of microorganisms are encountered. Polyhexanide is an antiseptic on the basis of polyhexamethylene biguanide (PHMB).

The vitamin preparation may contain further active agents beneficial in the treatment of the skin. An example in this context is dexpanthenol which is also a vitamin precursor. In the body, this agent is converted into pantothenic acid which is a vitamin stemming from the group of the B-vitamins (vitamin B5).

Dexpanthenol, for example, is contained in the preparation in an amount of between 1000 mg and 10000 mg per 100 g. If thought expedient, additional constituents may be polyethylene glycol 400 as well as sodium ascorbate as antioxidant.

The formulation may contain further substances which have proven their worth in the treatment of skin diseases. Examples in this context are taurine, caffeine, lysine, creatine, but also zinc salts, for example zinc sulfate. Especially zinc sulfate is a long-known agent used for the treatment of eczema and may, for instance, be present in the preparation in an amount of between 50 and 1000 mg/100 g.

The vitamin preparation proposed by the invention has preferably the following composition:
50 to 500 mg of polyhexanide in 20% aqueous solution
20 to 500 mg of poloxamer
1000 to 5000 mg of glycerin
500 to 5000 mg of hydroxyethyl cellulose 400
2500 to 7000 mg of urea
citric acid for buffering to pH 5.4
NaOH for buffering to pH 5.4 as well as
demineralized water ad 100 g.

The inventive vitamin preparation may be applied to the skin as a gel but may also be contained in gel form in a plaster/band-aid or pad which is then applied to the skin. Said preparation may also be administered or applied via sprays or as a tincture, for example in the form of nasal spray or eyedrops. Besides being effective in treating dermatoses and eczema, it was found that the preparation had a favorable effect on other types of skin and mucous membrane irritations, in particular on allergy-related irritations such as pollen allergies, house-dust allergy, and allergic reactions due to insect stings/bites.

The invention is explained in more detail by way of the following examples:

### EXAMPLE 1

A vitamin preparation in gel form was produced from the following constituents:

| | | |
|---|---|---|
| 1. | Vitamin B12 (cyanocobalamin) | 70 mg |
| 2. | Polyhexanide solution concentrate 20% (m/V) | 200 mg |
| 3. | Poloxamer 407 | 110 mg |
| 4. | Glycerin | 2500 mg |
| 5. | Hydroxyethyl cellulose 400 | up to 5000 mg, |
| | depending on the desired consistency | |
| 6. | Urea | 5000 mg |
| 7. | Citric acid | 300 mg |
| | for buffering to pH 5.4 | |
| 8. | If expedient, NaOH for buffering to pH 5.4 | |
| 9. | Demineralized water | ad 100 g |

The constituents are moderately heated and mixed and then stirred to produce a gel.

### EXAMPLE 2

The preparation listed in Example 1 is supplemented by 5000 mg of dexpanthenol and 2000 mg of PEG400.

Further constituents may be admixed, for example 300 mg of taurine, 25 mg of caffeine, 1000 mg of lysine, 500 mg of zinc sulfate, and/or 500 mg of creatine. Sodium ascorbate is added in case an oxidation inhibitor is needed.

Test

Cleaned pig skin is divided and coated with a vitamin B12 solution with 28 mg Co/l. After 1 h the skin is rinsed off, dried, shredded and extracted with aqueous hydrochloric acid in an ultrasonic bath.

The entraining agents used were 40 mg of Tween®, 100 mg poloxamer 180 and a combination of 100 mg poloxamer plus 40 mg Tween®, each for 100 ml of the test solution. Regarding the cobalt absorption into the skin the following values were obtained:

| | | 100 mg Poloxamer plus |
|---|---|---|
| 40 mg of Tween ® | 100 mg poloxamer | 40 mg Tween ® |
| 4.50 mg Co/kg | 5.40 mg Co/kg | 6.20 mg Co/kg |
| 100% | 120% | 138% |

With the pure vitamin B12 solution (without entrainer) a Co-absorption of only 3.0 mg Co/kg was obtained.

1. Transdermal vitamin preparation, for application to the skin, containing
a physiologically effective amount of vitamin B12,
a poloxamer as entrainer as well as
customary carrier substances for the formation of a carrier matrix.

2. Vitamin preparation according to claim 1, characterized in that the poloxamer is poloxamer 188 or 407.

3. Vitamin preparation according to claim 1, characterized in that it contains 40 to 100 mg of vitamin B12 per 100 g of the preparation.

4. Vitamin preparation according to claim 1, characterized in that it contains 50 to 500 mg of poloxamer per 100 g of the preparation.

5. Vitamin preparation according to claim 1, characterized in that said preparation contains polyhexanide.

6. Vitamin preparation according to claim 5, characterized in that it contains 50 to 500 mg of a 20% polyhexanide solution per 100 g of the preparation.

7. Vitamin preparation according to claim 1, characterized in that said preparation contains urea.

8. Vitamin preparation according to claim 1, characterized in that said preparation contains a polysorbate.

9. Vitamin preparation according to claim 8, characterized in that it contains 20 to 100 mg of polysorbate per 100 g of the preparation.

10. Vitamin preparation according to claim 8, characterized in that said preparation contains polysorbate 20 (Tween®).

11. Vitamin preparation in gel form according to claim 1, characterized in that said preparation contains
40 to 100 mg of vitamin B12 or of another precursor of coenzyme B12
50 to 500 mg polyhexanide in 20% aqueous solution
20 to 500 mg of poloxamer
1000 to 5000 mg of glycerin
500 to 5000 mg of hydroxyethyl cellulose 400
2500 to 7000 mg of urea
citric acid for buffering to pH 5.4
NaOH for buffering to pH 5.4 as well as
demineralized water ad 100 g.

12. Vitamin preparation according to claim 11, characterized in that it additionally contains 1000 mg to 4000 mg of PEG400.

13. Vitamin preparation according to claim 1, contained on, in, or on and in a pad for application to the skin, as nasal spray or tincture for application to mucous membranes.

14. Use of poloxamers as entraining agents for administering physiologically and/or therapeutically active and effective substances through the skin.

15. Vitamin preparation according to claim 1 in gel form.
